(19) United States
(12) Patent Application Publication
(10) Pub. No.: US 2025/0134458 A1
(43) Pub. Date: May 1, 2025

(54) BIOMECHANICAL ALIGNMENT VERIFICATION SYSTEM FOR AN ASSISTIVE MEDICAL DEVICE

(71) Applicant: Assistive Technology Development, Inc., Loveland, CO (US)

(72) Inventors: Todd William Roberts, Wilmington, CA (US); Bianca Lisa Riello, Pittstown, NJ (US); Owen P. Kent, Loveland, CO (US); Pablo David Amor Lacaba, Brussels (BE); Grace O'Connell, Berkeley, CA (US); Charlotte Adiaz Lao, Menlo Park, CA (US); Saahil Jayesh Patel, Vacaville, CA (US); David Day Wilkinson, Dedham, MA (US); Horacio Alain Millan, Calexico, CA (US); Tylor W. Langford, Trabuco Canyon, CA (US)

(21) Appl. No.: 18/837,149

(22) PCT Filed: Feb. 22, 2023
(86) PCT No.: PCT/US2023/062979

§ 371 Date: Aug. 8, 2024

Related U.S. Application Data
(60) Provisional application No. 63/313,714, filed on Feb. 24, 2022.

(51) Int. Cl. A61B 5/00; A61B 5/107

(52) U.S. Cl. CPC A61B 5/6812; A61B 5/1071

(57) ABSTRACT

An assistive medical device includes a brace configured to fit around an anatomical joint of a user. One or more sensors, such as an inertial measurement unit and an angle sensor, determine pose (e.g., position and/or orientation) of the brace. A controller receives data from the sensors and compares the data from the sensors to a pose of the anatomical joint that is either measured (e.g., via separate limb pose sensors) or assumed (e.g., by instructing the user to assume a specific pose). Based on the comparison, an indication of the alignment status between the brace and the limb is determined to guide an alignment verification process.

### CROSS-REFERENCE TO RELATED APPLICATIONS

This application claims the benefit of U.S. Provisional Application No. 63/313,714, filed Feb. 24, 2022, which is incorporated by reference in its entirety.

### BACKGROUND

### Technical Field

The described embodiments relate to an alignment verification system intended to detect and verify biomechanical alignment between an assistive device and an anatomical joint.

### Related Art

Conventional assistive devices, such as exoskeletons, orthoses, and prostheses rely on proper alignment between the device and anatomy to achieve the full benefits of the device. However, outside of clinical settings, achieving appropriate alignment is often challenging and may result in devices being used improperly.

### BRIEF DESCRIPTION OF THE DRAWINGS

FIG. 1 is a first example embodiment of a powered orthosis system including a first example embodiment of a powered orthosis device.

FIG. 2 is a block diagram of electronics for a powered orthosis device.

FIG. 3 is an example structure for a powered orthosis device showing placement of sensors.

FIG. 4 illustrates example rotational axes associated with a powered orthosis device.

FIG. 5 is a second example embodiment of a powered orthosis device.

FIG. 6 is a third example embodiment of a powered orthosis device.

FIG. 7 is a fourth example embodiment of a powered orthosis device.

FIG. 8 is an example embodiment of a powered orthosis device with an alignment element.

FIG. 9 is a flowchart of an embodiment of a method for aligning a brace with a joint of a user.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures.

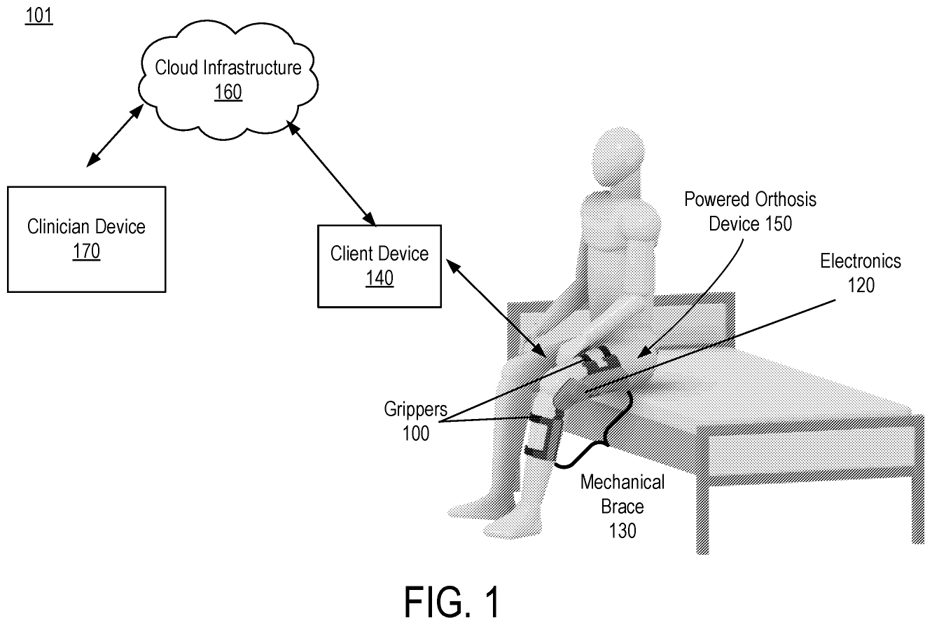

FIG. 1 illustrates an example embodiment of a assistive medical system 101 including a powered orthosis device 150 and supporting infrastructure (e.g., client device 140, cloud infrastructure 160, and clinician device 170). The powered orthosis device 150 provides assistive torque, resistive torque, or passive support to a joint of a user while collecting and reporting various metrics that may facilitate monitoring of the patient's condition and rehabilitation exercises. Example metrics that may be monitored include, for example, range of motion (ROM), strength, and walking gait data. The assistive medical system 101 may furthermore facilitate various exercises (which may be customized to specific patients) based on the monitored metrics. The assistive medical system 101 may be utilized in an at-home environment (potentially with support from a remote clinician) and/or in a clinical setting.

The powered orthosis device 150 includes a mechanical brace 130 with grippers 100 and various electronics 120. The orthosis device 150 communicates with a telehealth application executing on a client device 140 (e.g., a mobile phone, tablet, or computer) that enables communication with a remote care team (via a clinician device 170) and a cloud infrastructure 160 that provides cloud-based storage and processing for data collected by the powered orthosis device 150. The telehealth application may furthermore enable the patient to access various data, analytics, or controls relating to operation of the powered orthosis device 150.

The assistive medical system 101 may be utilized in telerehabilitation applications by enabling features such as remote patient monitoring by clinicians, communicating rehabilitation plans based on monitored performance, and performing various advanced analytics that may aid in rehabilitation. Furthermore, the assistive medical system 101 may provide localized feedback and control by the patient, without necessarily involving a remote clinician.

The powered orthosis device 150 illustrated in FIG. 1 is representative of an example structure for supporting a knee joint. This type of powered orthosis device 150 may be utilized following an orthopedic surgical procedure such as total knee arthroplasty or anterior cruciate ligament repair. In another example, the orthosis device 150 may instead be intended for supporting the elbow of a user following a surgery for a condition such as lateral epicondylitis. In further examples, the powered orthosis device 150 may be configured to support other joints of a user such as an ankle, wrist, shoulder, hip, toe joint, finger joint, neck, back, or other joint.

The mechanical brace 130 comprises a biomimetic mechanical assembly that mimics and supports the anatomical joint of the user. For example, the mechanical brace 130 comprises a pivot point that connects a lower brace (attached to the limb via a lower gripper 100) and an upper brace (attached to the limb via the upper gripper 100). The mechanical brace 130 may be configured to provide resistive torque, assistive torque, or a combination thereof to the anatomical joint it supports by applying torque to the pivot point as further described herein. In further embodiments, the orthosis device 150 may comprise a passive device that is not necessarily powered to actively provide resistive or assistive torque. Additionally, the orthosis device 150 may switch between operating in passive, assistive, and resistive modes.

The one or more grippers 100 each serve as an attachment point for attaching the brace 130 to a limb of the patient. The gripper 100 may be patient-specific and may be structured to conform to a unique anatomy of a patient via a custom molding and/or sizing process. Alternatively, in some implementations, a universal gripper 100 design may be used, such as a flexible strap. In the example orthosis device 150 depicted in FIG. 1, the device 150 includes two grippers 100, one above the anatomical joint (in this example, the knee) and one below the joint. Alternative embodiments may include different numbers and positions of grippers 100 appropriate for the joint being supported.

In some embodiments, the gripper 100 may comprise an adjustable structure that is configured to wrap around a portion of a limb and is capable of being tightened or loosened. In some embodiments, the gripper 100 includes a dial, a ratchet, or another controllable tensioning mechanism for manually controlling tension. Furthermore, in some embodiments, the gripper 100 may include a tension sensor that senses the tension level. In other embodiments, the gripper 100 includes an electric actuator that adjusts tension of the gripper 100 around the limb in response to one or more electronic control signals.

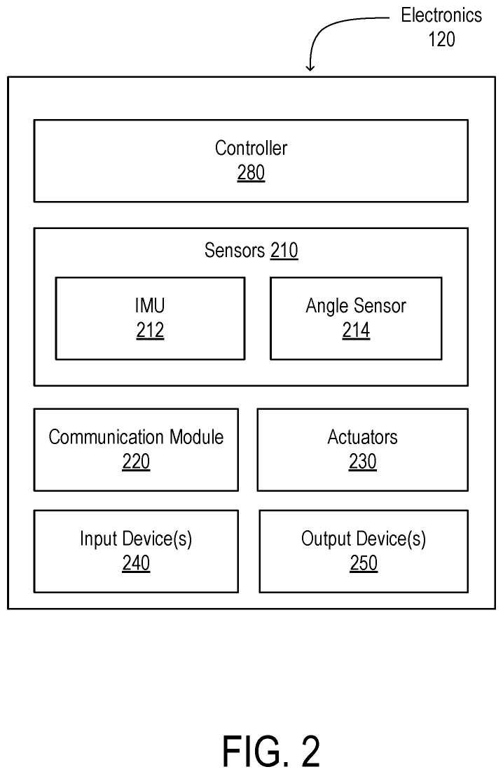

The electronics 120 include various components for sensing characteristics of the orthosis device 150, controlling operation of the device 150, receiving various inputs, and generating various outputs associated with the operation of the orthosis device 150. An example architecture for the electronics 120 is illustrated in FIG. 2 and described in further detail below.

The client device 140 and the clinician device 170 each comprise at least a processor and non-transitory computer-readable storage medium storing instructions for performing functions such as collecting data from the powered orthosis device 150, processing the data, and presenting the data. For example, the client device 140 may execute an application that facilitates an alignment process of the powered orthosis device 150 and may guide the user through various exercises with support from the powered orthosis device 150. The client device 140 may furthermore collect various sensor data from the powered orthosis device 150 and present it as feedback to the user and/or provide to the clinician device 170 for remote monitoring. In further embodiments, the client device 140 may generate control signals to control various functions of the powered orthosis device 150.

The clinician device 170 may execute a clinician application that may include similar functionality to the client device 140 and/or other functions specific to a clinician. For example, the clinician device 170 may present interfaces associated with remote monitoring of different patients, programming exercises for different patients, and/or providing other functions relating to the clinician's role.

The client device 140 and clinician device 170 may operate in conjunction with the cloud infrastructure 160 (e.g., via a network) to communicate relevant information between the devices 140, 170 and to perform various processing functions that may be offloaded by the client device 140 or clinician device 170. For example, the cloud infrastructure 160 may perform various advanced analytics algorithms based on data obtained from the client device 140 relating to operation of the powered orthosis device 150. These analytics algorithms may be user-specific (i.e., based only on data from a single user) or may be based on data obtained from a population of users. The cloud infrastructure 160 may include one or more physical servers, virtual servers, or a combination thereof and one or more storage mediums storing instructions for supporting functionality of the client device 140 and clinician device 170.

FIG. 2 is a block diagram of electronics 120 that may be integrated in the assistive medical system 101. The electronics 120 may include a controller 280, one or more sensors 210, a communication module 220, one or more actuators 230, one or more input devices 240, and one or more output devices 250. In other embodiments, the electronics 120 may include different or additional components.

The sensors 210 may comprise brace pose sensors that sense a pose of the mechanical brace 130 including its orientation and/or position. The sensors 210 include an internal measurement unit 212, an angle sensor 214, and/or other sensors. In the example structure of FIG. 1, a single IMU 212 may be positioned on the mechanical brace 130 on one side of a joint (e.g., above the knee). The IMU 110 may include one or more gyroscopes, one or more accelerometers, one or more magnetometers, or a combination thereof. The IMU 110 generally generates sensor data indicative of the orientation of the IMU relative to gravity. In various embodiments, when the orthotic device is properly aligned, the IMU 110 is positioned such that an axis of an accelerometer of the IMU 110 is substantially parallel to a long axis of an underlying bone structure of the user's limb in the sagittal plane.

The angle sensor 214 may be positioned proximate to the joint of the mechanical brace 130 and is aligned proximate to the user's joint when the orthotic device 150 is properly positioned. For example, the angle sensor 214 is positioned to enable detection of the joint angle. In some embodiments, the angle sensor 214 is a rotary encoder. In other embodiments the angle sensor 214 is a potentiometer, a twin-axis goniometer, or other device that converts mechanical rotation into an electrical signal.

The actuators 230 may generate assistive and/or resistive torque applied to the joint of the mechanical brace 130. The actuators 230 may include a power supply and a motor that applies torque in response to one or more control signals from the controller 280 as further described below. In the example structure of FIG. 1, one or more actuators 230 may be positioned proximate to the joint to apply torque between the upper and lower portions of the mechanical brace 130.

In some embodiments, one or more additional actuators 230 may be coupled to the grippers 100 to actively control tension of the gripper 100 around a user's limb in response to a control signal. For example, a control signal may cause an actuator to increase tension, decrease tension, or set the tension to a specific level.

The controller 280 controls the powered orthosis device 150, facilitates various data processing functions, and facilitates communication with the external client device 140 and/or clinician device 170. In an embodiment, the controller 280 may operate in at least an operational mode and an alignment verification mode. In the operational mode, the controller 280 controls the actuators 230 to cause the brace 130 to provide resistive and/or assistive torque to the relevant joint. In some applications, the controller 280 may operate in conjunction with the client device 140 to guide the patient through different exercises that may involve applying varying levels of torque while a user interface of the client device 140 (or a human clinician) guides the user through various poses and motions. In other applications, the controller 280 may control the device 150 to apply fixed levels of torque that may be controllable by a patient (e.g., via the client device or an input device 240 of the powered orthosis device 150 itself) and/or a clinician (e.g., via the clinician device 170). In a passive mode, the controller 280 may deactivate actuators 230 and provide only passive support.

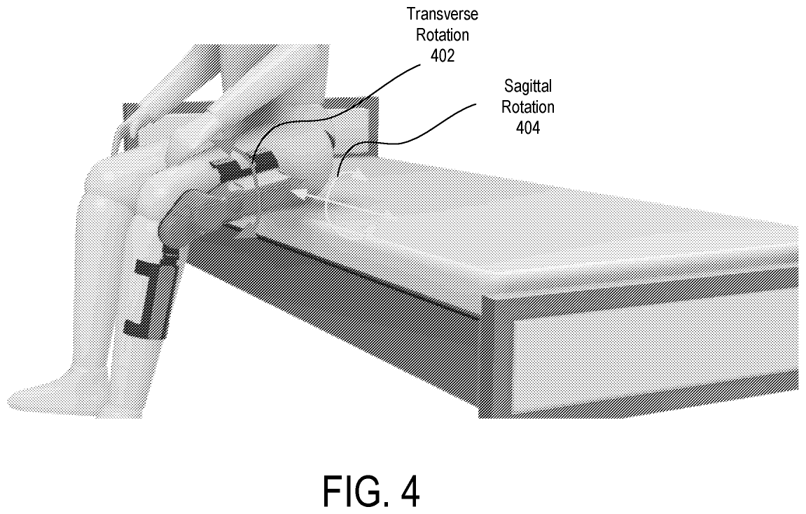

In the alignment verification mode, the controller 280 operates to detect an alignment of the orthosis device 150 relative to the user's limb based on data from the sensors (e.g., the IMU 212 and the angle sensor 214) and facilitate positioning to achieve proper alignment. For example, the patient may be instructed (by the client device 140 or a clinician) to assume a particular pose, such as sitting with the upper leg horizontal and the lower leg at an approximately ninety degree angle. The controller 280 may then obtain sensor data and detect alignment when the sensor data meets certain predefined alignment conditions associated with a predefined pose. For example, the alignment conditions may be satisfied when the IMU 212 indicates a substantially horizontal orientation relative to the ground (i.e., within a predefined error range) and the angle sensor 214 indicates a substantially ninety degree angle of the joint (i.e., within a predefined error range). The specific alignment criteria may vary in different embodiments to enable detection of the alignment of the status when the user assumes different predefined poses. In an example implementation, alignment may be evaluated relative to reference angles about the transverse and sagittal axes, as further explained with reference to FIG. 4. Below. In further embodiments, the controller 280 may detect alignment status associated with a sequence of different predefined poses.

In alternative embodiments, the orientation of the mechanical brace 130 may instead be determined from a vision-based tracking system that includes a camera and image processing system. The vision-based tracking system extracts consecutive images and analyzes the images to estimate joint angles using a two-dimensional analysis. In an embodiment, the vision-based system includes one camera with at least two perspectives to triangulate two-dimensional information into three-dimensional coordinates. For two-dimensional analysis, estimates are based on two known points on a limb segment that may be established via marker-based systems and associated markers or via markerless systems and associated body models. In this embodiment, the sensors 210 of the orthosis device 150 may be optionally omitted.

The controller 280 may characterize the alignment status in various ways. For example, in one embodiment, the controller 280 may characterize the alignment in a binary manner as either aligned or misaligned. Alternatively, the controller 280 may characterize the alignment status as one of three or more predefined states (e.g., significantly misaligned, slightly misaligned, and aligned) that correspond to different ranges of an alignment metric characterizing alignment magnitude. Furthermore, when the controller 280 detects misalignment, it may generate an output (e.g., via an output device 250) indicative of the direction of misalignment (or conversely, the direction of motion needed to correct misalignment). For example, the controller 280 may indicate whether the device 150 should be rotated clockwise or counterclockwise about one or more reference axes, or whether it should be laterally shifted in a particular direction to achieve improved alignment.

The controller 280 furthermore facilitates providing feedback to the user regarding the detected alignment status to assist the user in properly positioning the powered orthosis device 150. For example, the controller 280 may transmit an alignment signal to the client device 140 indicative of the alignment state and enable the client device 140 to output information indicative of the sensed alignment state. Alternatively, or in addition, the controller 280 may control an output device 250 (e.g., a visual indicator such as a light emitting diode (LED), a speaker, or a haptic feedback device) integrated into the orthosis device 150 to provide output indicative of the alignment state. For example, the controller 280 may control an LED of the orthosis device 150 to display varying colors or blinking patterns to denote different alignment states (e.g., red for significantly misaligned, yellow for slightly misaligned, and green for aligned). Alternatively, or in addition, a haptic device to vibrate according to different patterns and/or a speaker to output audible signals indicative of the alignment statement. The alignment state may furthermore be displayed in a user interface of the client device 140 using various visual elements.

In other embodiments, the controller 280 does not necessarily perform the alignment determination locally, and instead sends raw or filtered sensor data to the client device 140. The client device 140 may then perform the alignment detection according to any of the techniques described above. The client device 140 may directly display information indicative of the alignment state and/or may send an alignment signal to the controller 280 to enable feedback via an output device 250 of the orthosis device 150. In yet further embodiments, the controller 280 may compute the alignment status locally and send both an alignment signal and various raw or filtered sensor data to the client device 140. This embodiment may enable alignment detection without necessarily relying on a connection to the client device 140 while still enabling the client device 140 to facilitate various analysis and presentation of the sensor data when a connection is available.

The controller 280 may furthermore generate various control signals that control the actuators 230 of the orthosis device 150 in response to detecting the alignment state. For example, the controller 280 may control a tensioning mechanism to automatically configure a tension level of the gripper 100 when misalignment is detected to relatively lower tension to enable repositioning of the brace 130. When alignment is detected, the controller 280 may automatically control an actuator 230 of the tensioning mechanism to automatically tighten the gripper 100 to an operational tension level that secures the orthosis device 150 in the aligned position.

In an embodiment, the controller 280 may initially operate in the alignment verification mode until proper alignment is detected. Upon detection, the controller 280 may automatically switch to the operational mode. In some embodiments, the controller 280 may periodically reenter the alignment verification mode from the operational mode to recheck the alignment. Furthermore, the controller 280 may automatically switch from the operational mode to the alignment verification mode when a condition is detected that is indicative of misalignment.

In other embodiments, the controller 280 may detect a manually set tensioning level of the gripper 100 (via a tension sensor) and set the controller mode to the alignment verification mode or the operating mode dependent on the detected tension level. For example, when the gripper 100 is sufficiently loosened to enable repositioning of the orthosis device 150, the controller 180 may automatically switch to an alignment mode that performs alignment verification as described above. When the controller 280 detects manual tensioning of the gripper 100 to a tension level suitable for operation of the orthosis device 150, the controller 280 may automatically switch to an operating mode to facilitate various exercises or other functions of the orthosis device 150.

In further embodiments, control signals for switching between the alignment verification mode and the operating mode may be generated by the client device 140 and/or the clinician device 170 and communicated to the controller 180. Here, the mode may be set via a user interface of the client device 140 or clinician device 170, or may be set automatically via various sensed conditions. In embodiments, the controller 280 may coordinate with the client device 140 and/or clinician device 170 to communicate the mode (which may be set by either the controller 280, the client device 140, or the clinician device 170). The client device 140 may then output guidance via a user interface to help facilitate the alignment process. For example, the client device 140 may instruct the user to assume a particular pose during alignment verification, and the controller 280 may then detect the alignment relative to that pose.

In an embodiment, the controller 280 may be implemented as a processor and a storage device. The storage device may include a non-transitory computer readable storage medium having instructions encoded thereon that, when executed by the processor, cause the processor to perform various functions attributed to the controller 280 herein. The controller 280 may furthermore include storage for storing various data described herein such as acquired sensor data and predefined poses for comparison during the verification process.

The communication module 220 comprises a wired or wireless communication interface for coupling the controller 280 to the client device 140, the clinician device 170, and/or the cloud infrastructure 160. The communication module 220 includes a wireless transceiver for transmitting and receiving data through one or more wireless communication protocols. Example wireless communication protocols include 802.11, 4G, 5G, code division multiple access (CDMA), BLUETOOTH®, or other wireless communication protocols. The communication module 220 may furthermore include a wired transceiver for transmitting and receiving data using one or more wired communication protocols. Examples of wired communication protocols include Ethernet, Universal Serial Bus (USB), or other protocols for exchanging data through a wired connection. In various embodiments, the communication module 220 includes support for both wired connections and wireless connections. The communication module 220 may provide application programming interface (API) functionality to send data directly to native client device operating systems, such as IOS® or ANDROID™. The communication module 220 may furthermore facilitate communication between various electronics 120 of the powered orthosis device 150 using protocols such as Inter-Integrated Circuit (I2C), controller area network bus (CAN bus), universal asynchronous receiver-transmitter (UART), or other protocols.

The input device 240 may comprise one or more control elements that may be integrated with the powered orthosis device 150 to facilitate various functions. The input device 240 may comprise, for example, one or more buttons, dials, switches, touchscreens, or other devices for providing user inputs. The input device 240 may be used to provide functions such as turning the device 150 on or off, switching modes, setting configuration parameters, or other functions.

The output device 250 may include visual, audio, or haptic devices for providing one or more output signals. For example, the output device 250 may provide feedback indicative of the alignment state as described above and/or may provide various other status information in response to inputs via the input device 240 and/or the client device 140.

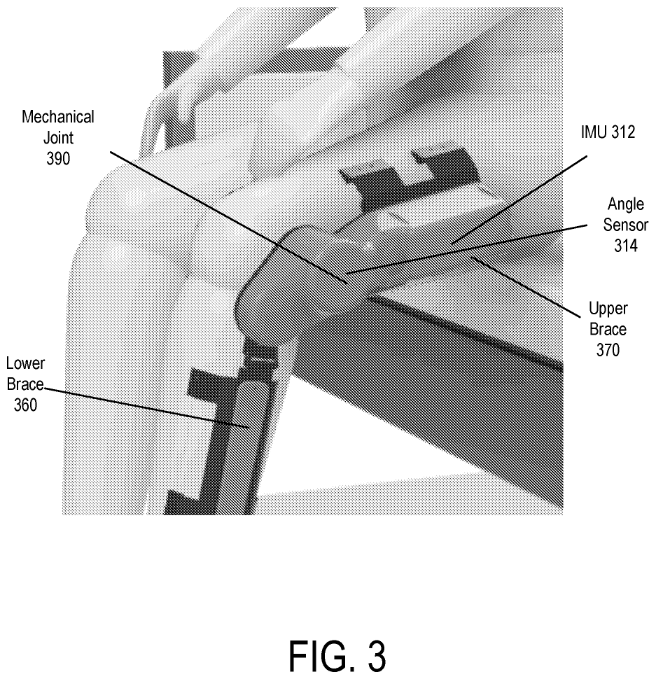

FIG. 3 illustrates an example structure for a powered orthosis device 150 showing placement of the sensors 210. In this example, the mechanical brace 130 includes an upper brace 370 above a mechanical joint 390 and a lower brace 360 below the mechanical joint 390. When aligned, the mechanical joint 390 will be approximately aligned with the anatomical joint of the limb. In this example, the device 150 includes an IMU 312 that is integrated within a housing structure of the upper brace 370 and an angle sensor 314 that is integrated within a housing structure proximate to the mechanical joint 390. In alternative embodiments, the sensors 210 may be placed in different locations. Furthermore, in alternative embodiments, the sensors 210 may be on an external surface of the brace 310 without necessarily being integrated within a housing.

FIG. 4 illustrates axes of rotation that can be checked during the alignment verification process. Here, the alignment verification process may sense transverse rotation 402 about a transverse access (parallel to the limb) and sagittal rotation 404 about a sagittal axis (perpendicular to the limb) relative to respective reference angles. The alignment verification process may sense relative misalignment of the orthotic device with respect to both transverse and sagittal reference angles and provide feedback to enable the user to correct the alignment.

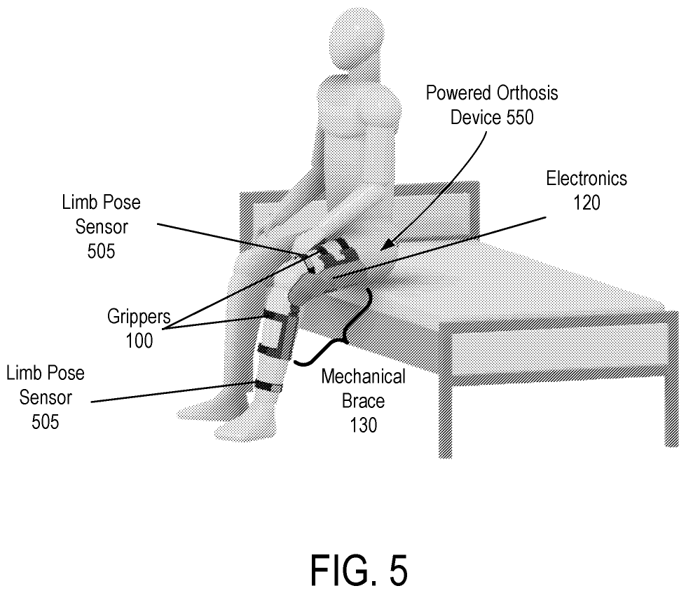

FIG. 5 shows an alternative example of a powered orthosis device 550. The powered orthosis device 550 is similar to the powered orthosis device 150 described above, but additionally includes one or more independent limb pose sensors 505 configured to determine an orientation of the anatomical joint of the user independently of the orientation of the mechanical brace 130. The limb pose sensors 505 may comprise one or more inertial measurement units (IMUs) or other orientation and/or position sensors located on each side of the anatomical joint of the user (e.g., above and below the knee in the example of FIG. 5).

The limb pose sensors 505 may be mounted to the limb of the user (e.g., using a strap or other attachment mechanism) with an axis of an accelerometer of an limb pose sensor 505 parallel to a long axis of an underlying bone structure in the sagittal plane of the limb of the user. Such a configuration allows the limb pose sensors 505 to be placed anywhere along a segment of the user's limb being measured.

In this embodiment, the alignment state of the mechanical brace 130 with respect to the user's joint can be assessed by calculating the relative pose of the joint from the limb pose sensors 505 and the pose of the mechanical brace 130 from the sensors 210. The misalignment between the relative poses may then be characterized to determine the alignment state. In this embodiment, the alignment state may be detected while the user assumes any arbitrary pose and does not necessarily depend on comparing the sensed orientation of the orthosis device 150 to a predefined pose assumed by the patient.

In another embodiment, the pose of the anatomical joint of the user may be determined using a vision-based tracking system. In this embodiment, the limb pose sensors 505 may be omitted, and the relative orientation between the brace 130 and the limb is determined by comparing the detected pose of the brace 130 with the pose of the patient derived from the vision system. In other embodiments, orientation of both the user's joint and the mechanical brace 130 are determined from a vision-based tracking system. In yet further embodiments, a vision-based tracking system is combined with the sensors 210 in the brace 130 and/or limb pose sensors 505 attached to the limbs for alignment verification.

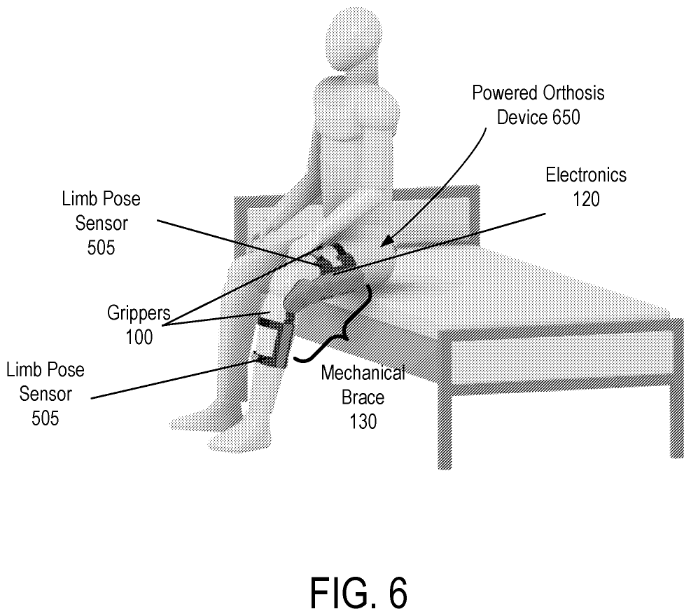

FIG. 6 illustrates another example embodiment of a powered orthosis device 650. In this embodiment, the limb pose sensors 505 may be physically attached to the mechanical brace 130 instead of being physically separate components. The attachment mechanism may be configured such that the limb pose sensors 505 can be positioned at least semi-independently relative to the mechanical brace 130 and may move with the user's limb. For example, a limb pose sensor 505 may be coupled to a flexible strap that is wrapped around the user's limb and attaches to mechanical brace 130 at a connection point. This enables the limb pose sensors 505 to sense of the limb pose independently of the mechanical brace 130, while maintaining a physical connection so that the user need not keep track of multiple independent components.

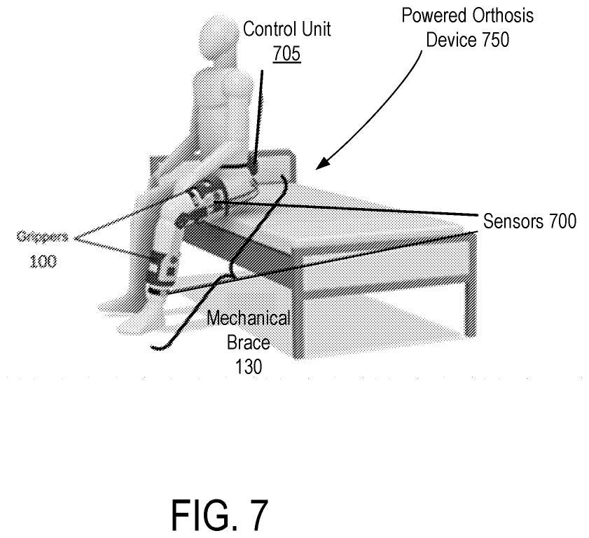

FIG. 7 illustrates another example embodiment of a powered orthosis device 750. In this embodiment, the electronics 120 include a control unit 705 that may be physically separated from the mechanical brace 130. Sensors 700 may be positioned on the brace 130 and coupled via wires or wirelessly to the control unit 705 which may house the controller 280, communication module 220, and other electronics 120. The control unit 705 may be structured to be mountable to the patients back, similar to a backpack. Alternatively, the control unit 705 may comprise a different structure that is not necessarily worn by the patient.

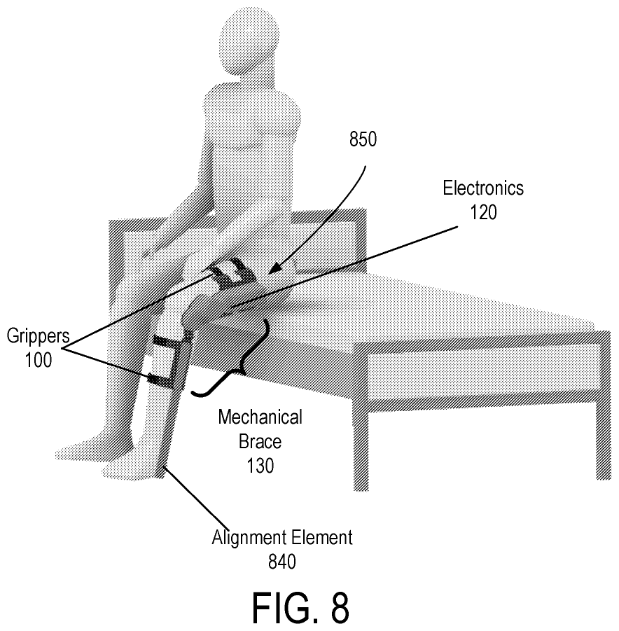

FIG. 8 illustrates an embodiment of a orthosis device 850 that includes an alignment element 840 for physically guiding alignment of the mechanical brace 130. In the illustrated example, the alignment element 840 extends from a lower portion of the brace 130 on the lower leg and under the foot. The alignment element 840 may be adjustable in height and may be substantially flush with the bottom of the foot when the brace 130 is properly aligned. In an example use case, the orthosis device 850 may be aligned using the alignment verification process described above. Once aligned, the alignment element 840 may be extended to the bottom of the foot and secured at the selected height. During future use of the orthosis device 850, the patient can easily position the brace 130 at the appropriate position and orientation by relying on the physical guidance from the alignment element 840 without necessarily having to repeat the alignment verification process.

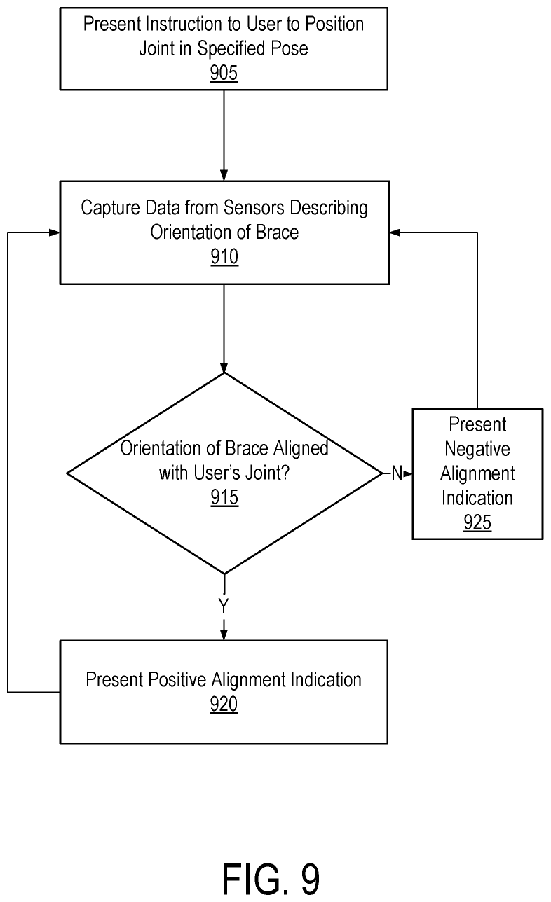

FIG. 9 is a flowchart of an example embodiment of a method for aligning a mechanical brace 130 with a joint of a user. A client device 140 presents 905 an instruction to a user to position a joint in a specified pose. For example, a display of the client device 140 presents a user interface that may illustrate an image of the specified pose and an instruction for the user to replicate the displayed image of the specified pose. In another example, the client device 140 plays audio or text-based prompts to the user identifying the specified pose. The instructions may be presented when the orthosis device 150 (and associated client device 140) operates in an alignment mode for the purpose of performing alignment verification.

The controller 280 captures 910 data from one or more sensors 210 indicative of the pose of the orthosis device 150 as described above. The controller 280 determines 915 whether the orientation of the mechanical brace 130 of the powered orthosis device 150 is aligned with the joint of the user. For example, the controller 280 determines whether the sensed data meets specified alignment criteria such as being within a threshold range of the expected pose set forth in the user instructions. The controller 280 may compute an alignment metric that may be based on a series of sensor measurements over a predefined time period to determine the alignment status. In response to sensor data meeting the alignment criteria, the controller 280 determines 915 that the mechanical brace 130 is aligned with the joint of the user. Otherwise, in response to the sensor data failing to meet the alignment criteria, the controller 280 determines that the device 150 is misaligned. In embodiments where the powered orthosis device includes limb pose sensors 505 for independently detecting the limb pose, the controller 280 may instead compare the sensor data from the brace 130 to the sensor data from the limb pose sensors 505 to detect the alignment state.

In response to the alignment signal indicating sufficient alignment, a positive alignment indication may be presented 920 (e.g., via the client device 140, an integrated output device 250, or both). Otherwise, a negative alignment indication may be presented 925.

As described above, other embodiments may include presenting more granular information about the alignment state. For example, the client device 140 or other output device 250 may present a state indicative of how close the device 150 is to achieving proper alignment (e.g., using a color coded system or other scheme). Additionally, the client device 140 or other output device 250 may present a specific misalignment magnitude and/or direction, or may generate specific instructions for achieving proper alignment. Once appropriate alignment is achieved, the orthosis device 150 may transition to an operational mode for facilitating various rehabilitation exercises or performing other functions.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention.

What is claimed is:

1. An assistive medical device comprising:
a mechanical brace configured to be attached to a limb of a user having an anatomical joint, the mechanical brace including at least one mechanical joint supporting the anatomical joint;
one or more brace pose sensors configured to generate brace pose sensor data indicative of a pose of the mechanical brace;
a controller configured to capture the brace pose sensor data from the one or more brace pose sensors, to obtain a limb pose of the limb, to characterize an alignment status between the pose of the mechanical brace and the pose of the limb, and to generate one or more alignment signals indicative of the alignment status; and
an output device configured to output the alignment signal to indicate the alignment status.

2. The assistive medical device of claim 1, wherein the one or more brace pose sensors include an inertial measurement unit to sense an orientation of the mechanical brace relative to gravity, and an angle sensor coupled to the mechanical brace to measure an angle of the mechanical joint.

3. The assistive medical device of claim 1, wherein the controller is configured to characterize the alignment status by:
comparing the pose of the mechanical brace derived from the brace pose sensor data to a predefined pose of the anatomical joint that the user is instructed to assume to generate an alignment metric;
performing a comparison of the alignment metric to one or more thresholds; and
determining the alignment status based on the comparison.

4. The assistive medical device of claim 1, wherein the controller is configured to characterize the alignment status in a binary manner as either aligned or misaligned.

5. The assistive medical device of claim 1, wherein the controller is configured to characterize the alignment status as falling into one of three or more ranges indicating varying levels of alignment.

6. The assistive medical device of claim 1, wherein the controller is configured to characterize the alignment status as a numerical amount of misalignment according to magnitude, direction, or both.

7. The assistive medical device of claim 1, wherein the assistive medical device further comprises:
one or more limb pose sensors configured to couple directly to the limb to generate limb pose sensor data indicative of the pose of the limb independently of the pose of the mechanical brace.

8. The assistive medical device of claim 7, wherein the one or more limb pose sensors are physically separate from the mechanical brace.

9. The assistive medical device of claim 7, wherein the one or more limb pose sensors are physically coupled to the mechanical brace at an attachment point in a manner that enables freedom of motion of the mechanical brace relative to the limb pose sensors.

10. The assistive medical device of claim 7, wherein the controller is configured to characterize the alignment status by:
comparing the pose of the mechanical brace derived from the brace pose sensor data to the pose of the limb derived from the limb pose sensor data to generate an alignment metric;
performing a comparison of the alignment metric to one or more thresholds; and
determining the alignment status based on the comparison.

11. The assistive medical device of claim 1, wherein the mechanical brace comprises:
one or more grippers configured to wrap around the limb of the user to secure the mechanical brace to the limb, wherein the one or more grippers include adjustable tensioning mechanism for adjusting a tightness of the one or more grippers.

12. The assistive medical device of claim 11, wherein the adjustable tensioning mechanism comprises an electronically controlled actuator for adjusting the tightness in response to a tension control signal from the controller.

13. The assistive medical device of claim 12, wherein the controller is configured to control the electronically controlled actuator to automatically increase the tightness of the one or more grippers from an alignment verification level to an operational level in response to the controller characterizing the alignment status as meeting an alignment criteria.

14. The assistive medical device of claim 12, wherein the controller is configured to control the electronically controlled actuator to automatically decrease the tightness of the one or more grippers from an operational level to an alignment verification level in response to the controller detecting a misalignment condition.

15. The assistive medical device of claim 11, wherein the one or more grippers includes a tension sensor to obtain tension sensing data indicative of a tension level of the one or more grippers and send the tension sensing data to the controller, and wherein the controller is configured to detect that the tension level is indicative of an alignment being performed, and automatically generate the one or more alignment signals responsive to detecting the alignment is being performed.

16. The assistive medical device of claim 1, wherein following verification of the alignment status, the controller is configured to operate in an operational mode in which the controller operates in a passive mode or controls one or more actuators of the mechanical joint to provide assistive or resistive torque to the mechanical joint.

17. The assistive medical device of claim 16, wherein the controller is operatively coupled to a client device via a communication module to enable the controller to send the brace pose sensor data to the client device and to receive control signals associated with the operational mode from the client device.

18. The assistive medical device of claim 1, further comprising:
an alignment element adjustably extendable from the mechanical brace to physically guide alignment of the mechanical brace.

19. A method for performing an alignment verification in association with a orthosis device. the method comprising:
storing to a controller of the orthosis device, pose data indicative of a specified pose associated with a joint supported by the orthosis device, wherein a user of the orthosis device is instructed to assume the specified pose for determining the alignment verification;
sensing, by one or more brace pose sensors, brace pose sensor data indicative of a pose of a mechanical brace of the orthosis device;
characterizing, by the controller, an alignment status between the pose of the mechanical brace and the specified pose;
generating, by the controller, one or more alignment signals indicative of the alignment status; and
outputting, by an output module of the orthosis device, the one or more alignment signals indicative of the alignment status.

20. An assistive medical system comprising:
an assistive medical device comprising:
a mechanical brace configured to be attached to a limb having an anatomical joint, the mechanical brace including at least one mechanical joint;
one or more brace pose sensors configured to generate brace pose sensor data indicative of a pose of the mechanical brace;
a controller configured to capture the brace pose sensor data from the one or more brace pose sensors, to characterize an alignment status between the pose of the mechanical brace and a pose of the limb, and to generate one or more alignment signals indicative of the alignment status; and
a communication module configured to communicate the one or more alignment signals;
a non-transitory computer-readable storage medium storing instructions executable by a processor of a client device, the instructions when executed by the processor causing the processor to perform steps including:
obtaining the one or more alignment signals from the assistive medical device; and
presenting, in a user interface of the client device, one or more visual elements indicative of the alignment status obtained from the one or more alignment signals.
